(19) United States
(12) Patent Application Publication
(10) Pub. No.: US 2011/0052035 A1
(43) Pub. Date: Mar. 3, 2011

(54) Vessel Extraction Method For Rotational Angiographic X-ray Sequences

(75) Inventors: Klaus J. Kirchberg, Plainsboro, NJ (US); Wai Kong (Max) Law, Yuk Po Court (HK); Chenyang Xu, Berkeley, CA (US)

(73) Assignee: Siemens Corporation, Iselin, NJ (US)

(21) Appl. No.: 12/872,039

(22) Filed: Aug. 31, 2010

Related U.S. Application Data
(60) Provisional application No. 61/238,740, filed on Sep. 1, 2009.

(51) Int. Cl. G06K 9/00; A61B 6/00

(52) U.S. Cl. 382/132; 378/15

(57) ABSTRACT

A method (100) of blood vessel extraction for rotational angiographic X-ray sequences, comprising obtaining a 2.5D vesselness detection response in 3D (208). The method (100) utilizes the projection matrices to realize the correspondence among different image frames to extract low level image features for subsequent segmentation and 3D image reconstruction.

### CROSS-REFERENCE TO RELATED APPLICATIONS

This application claims the benefit of Provisional U.S. Patent Application Ser. No. 61/238,740 entitled, “Vessel Extraction Method For Rotational Angiographic X-Ray Sequences”, filed in the name of Klaus J. Kirchberg, Wai Kong (Max) Law, and Chenyang Xu on Sep. 1, 2009, the disclosure of which is also hereby incorporated herein by reference.

### FIELD OF INVENTION

The present invention relates to X-ray imaging. More particularly, the present invention relates to X-ray imaging techniques for coronary vessels.

### BACKGROUND OF THE INVENTION

The need for diagnostic imaging systems and methods for coronary disease has increased in recent years. 3D angiography is a relatively new imaging technique that may be implemented by a rotational X-ray imaging apparatus that acquires a series of 2D X-ray projections of the coronary area along an arced path. The rotation is accomplished by moving an X-ray source and an X-ray detector mounted on a rotatable C-arm about a patient. The X-ray detector converts the raw X-ray projections into image data signals for subsequent image processing by the X-ray imaging system.

Based on rotational X-ray imaging techniques, coronary arteries are visualized with the help of radio-opaque contrast agents administered to the vasculature of the patient. In this method, blood vessels filled with contrast agent appear darker than the neighboring regions within the patient in the X-ray images, i.e., the rotational series of 2D image data. To facilitate the diagnostic process, these contrast-enhanced images are commonly processed by computerized systems, including image processors, that form part of the overall X-ray imaging system. In particular, the computer processing segments the blood vessels from the X-ray angiograms (i.e., determines the boundaries between different portions of the image), and subsequently reconstructs a 3D image of the patient vasculature structure, also known as the coronary artery tree, which plays an important role in helping the clinician assess a patient's coronary condition.

To segment the blood vessels from the X-ray angiograms, one would consider the use of a vesselness measure (VM) during the processing, such as Frangi's vesselness measure (this is more fully described in a paper by A. Frangi, W. Niessen, and M. Viergever, entitled “Multiscale vessel enhancement filtering”, In: W. M. Wells, A. C. F. Colchester, S. L. Delp, The International Conference on Medical Image Computing and Computer Assisted Intervention 1998, LNCS, vol. 1496, pp. 130-137). A vesselness measure is used to examine how similar an imaged structure is to a tube, thus identifying a blood vessel. As a well-founded blood vessel detection approach, Frangi's vesselness method is widely applied in diagnostic imaging for dealing with various blood vessel detection problems. It is based on analyzing the second order intensity statistics in a multiscale fashion. Based on the Frangi's vesselness measure, there is a recent proposal to reconstruct the 3D vasculatures of an imaged patient by considering the 2D segmentation results obtained from two orthogonal image planes (this is described in a paper by A. Andriotis, A. Zifan, M. Gavaises, P. Liatsis, I. Pantos, A. Theodorakakos, E.•P. Efstathopoulos, D. Katritsis, entitled “A New Method of Three-dimensional Coronary Artery Reconstruction From X-Ray Angiography: Validation Against a Virtual Phantom and Multislice Computed Tomography”, Catheterization and Cardiovascular Interventions 2008, vol. 71, pp. 28-43). To further refine the reconstruction results, one can make use of all available image frames to reconstruct vascular trees (this is described in a paper by C. Blondel, G. Malandain, R. Vaillant, N. Ayache, entitled, “Reconstruction of Coronary Arteries From a Single Rotational X-Ray Projection Sequence”, IEEE Transaction on Medical Imaging 2006, vol. 25(5), pp. 653-663). This described method involves estimating the heart motion field to back project and align the coronary artery in different heart phases in order to maximize the number of usable image frames for reconstruction.

In a conventional coronary artery reconstruction routine, the 3D blood vessels are reconstructed by associating the 2D segmentation results of each individual image frame with the same heart phase. In the workflow of this reconstruction process, the 2D segmentation result of each image frame is first acquired. By making use of the available projection matrices, the subsequent reconstruction process attempts to displace the 2D segmented pixels in the reference image frame along the direction which is perpendicular to that image. It aims at obtaining 3D vessels that match the 2D segmentation results of different image frames obtained in different projection angles

However, due to the presence of various factors, for example, image noise, randomness of blood vessel intensity, overlapping of irrelevant structures, complicated blood vessel topology, partial volume effects and imaging artifacts, the segmentation results can be insufficient for reconstruction. Thus, there is a need to improve the segmentation quality. Considering the above-referenced blood vessel reconstruction approaches, a major drawback of present methods is that the correspondence between different image frames is not exploited during the segmentation process.

### SUMMARY OF THE INVENTION

The above problems are obviated by the present invention which provides a method of reconstructing 3D images of vascular structures, comprising obtaining 2D X-ray projection images of the vascular structures to be imaged; extracting image features from the X-ray images via the use of a 2.5D vesselness measure; segmenting the vascular structures from the X-ray images using the extraction results; and reconstructing 3D images of the vascular structures from the segmentation results. The vascular structures may comprise coronary arteries. The extracting step may be performed before the segmenting and the reconstructing steps, which may then also comprise applying an inverse radon transform on the X-ray images and performing vesselness detection to acquire vesselness responses. In such case, the applying and performing steps may be performed as one merged operation. Further, the performing step may comprise performing vesselness detection to acquire vesselness detection responses in 3D with the method comprising an additional step of resampling the vesseleness detection responses in 3D to acquire a vesselness detection response in 2D for each reference image frame, said segmenting step segmenting the vascular structures from the X-ray images using the resampling results.

Alternatively in such case, the performing step may comprise computing a Hessian matrix and obtaining vesselness measures using the inverse radon transform results. Then, the applying and performing steps may be performed as one merged operation. The performing step may then comprise performing vesselness detection to acquire vesselness detection responses in 3D with the method further comprising resampling the vesseleness detection responses in 3D to acquire a vesselness detection response in 2D for each reference image frame, said segmenting step segmenting the vascular structures from the X-ray images using the resampling results.

Alternatively, the extracting step may be performed before the segmenting and the reconstructing steps, which may then also comprise accumulating all the 2D X-ray projection images and performing vesselness detection on the accumulation results to acquire vesselness detection responses. In such case, the applying and performing steps may be performed as one merged operation. Then, the performing step may comprise performing vesselness detection to acquire vesselness detection responses in 3D with the method further comprising resampling the vesseleness detection responses in 3D to acquire a vesselness detection response in 2D for each reference image frame, said segmenting step segmenting the vascular structures from the X-ray images using the resampling results.

The present invention also provides a method of coronary artery 3D reconstruction, comprising obtaining a 2D X-ray projection sequence of a coronary artery to be imaged; and filtering each projection image of the back projection for the 2D X-ray projection sequence using a vesselness measure that realizes the correspondence among different image frames to extract low level image features for subsequent segmentation and image reconstruction of the coronary artery. The filtering step may comprise performing a merged operation of an inverse radon transform and a vesselness detection. Alternatively, the filtering step may comprise performing a merged operation of a filtered back-projected inverse radon transform and a vesselness detection. Alternatively, the filtering step may comprise performing a merged operation of an inverse radon transform, a Hessian matrix computation, and a vesselness measure. The method may also comprise resampling the filtering results to acquire a vesselness detection response in 2D for each reference image frame for subsequent 2D segmentation.

The present invention also provides a method of blood vessel extraction for rotational angiographic X-ray sequences, comprising obtaining a 2.5D vesselness detection response in 3D. In such case, the obtaining step may comprise utilizing the projection matrices to realize the correspondence among different image frames to extract low level image features for subsequent segmentation and 3D image reconstruction.

The present invention also provides a 3D X-ray imaging system, comprising an X-ray source that generates X-ray beams; an X-ray detector that is adapted to receive the X-ray beams; a support table positioned between the X-ray source and the X-ray detector such that the X-ray beams pass through a portion of the vasculature structure of a subject lying thereon and project onto the X-ray detector, said detector converting the raw X-ray projections into image data signals for subsequent processing; and a computer system which controls the operation of the system and its components and processes the image data obtained from the X-ray detector to transform them into a reconstructed volumetric image of the imaged portion of the vasculature structure for display, storage, and/or other usage. The computer system filters each projection image of the back projection for the X-ray images using a vesselness measure that realizes the correspondence among different image frames to extract low level image features for subsequent segmentation and 3D image reconstruction of the imaged portion of the vasculature structure. The system may further comprise a rotational X-ray apparatus whereby the X-ray source and the X-ray detector are mounted on opposite ends of, and coupled to one another via, a rotatable C-arm gantry arrangement that moves the X-ray source and the X-ray detector about the person and the table in a coordinated manner so that the X-ray projections of the imaged portion of the vasculature structure can be generated from different angular directions and a series of 2D X-ray projections are acquired along an arced path.

### DESCRIPTION OF THE DRAWINGS

For a better understanding of the present invention, reference is made to the following description of an exemplary embodiment thereof, and to the accompanying drawings, wherein:

FIG. 1 is a block diagram of an X-ray imaging system operable in accordance with the present invention;

FIG. 2 is a schematic representation of a blood vessel detection method implemented in accordance with the present invention;

FIG. 3 is a block diagram of different representations of the blood vessel detection method of FIG. 2;

FIG. 4 is a block diagram of an alternative method of blood vessel detection in accordance with the present invention.

### DETAILED DESCRIPTION

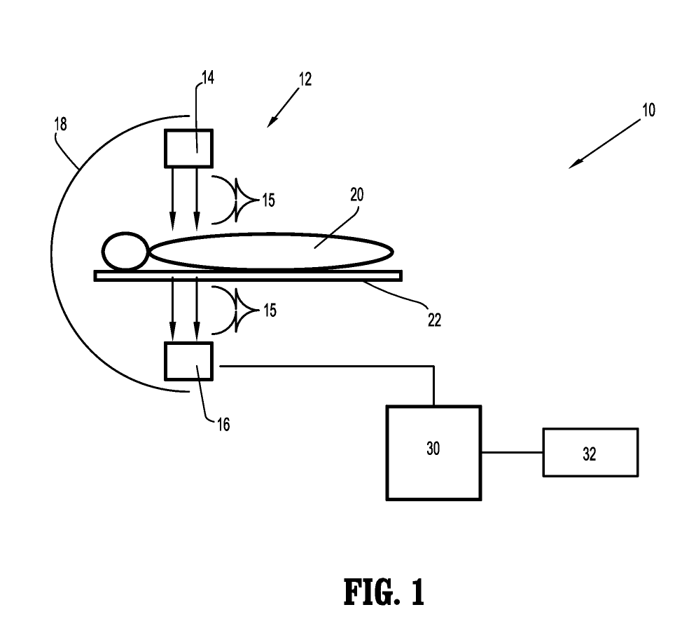

FIG. 1 is a block diagram of an X-ray imaging system 10 (simplified) that operates in accordance with the present invention. The system 10 comprises a rotational X-ray imaging apparatus 12 having an X-ray source 14 that generates X-ray beams 15 towards an X-ray detector 16. The X-ray source 14 and the X-ray detector 16 are mounted on opposite ends of, and coupled to one another via, a rotatable C-arm gantry arrangement 18. A patient to be imaged 20 is positioned on a support table 22 between the two components 14, 16 such that the X-ray beams 15 pass through the patient 20, and in particular, the coronary region of interest, and project onto the X-ray detector 16. The detector 16 converts the raw X-ray projections into image data signals for subsequent processing by the X-ray imaging system 10. As a result of the rotation of the C-arm 18, the X-ray source 14 and the X-ray detector 16 are moved about the patient 20 and the table 22 in a coordinated manner so that the X-ray projections of the vasculature structure of the patient 20 can be generated from different angular directions and a series of 2D X-ray projections of the coronary area are acquired along an arced path.

The rotational X-ray imaging apparatus 12 is operably coupled to a computer system 30 which controls the operation of the X-ray imaging system 10 and its components and processes the image data obtained from the X-ray detector 16 to transform them into a visual representation of the patient's vasculature structure (i.e., reconstructed images of the vasculature structure). In particular, the computer system 30 operates on the image data using well-known mathematical image processing and reconstruction algorithms/techniques, such as segmentation, Fourier transforms, etc., and generates for display, storage, and/or other usage corresponding X-ray images. The computer system 30 is also operably connected to appropriate user interfaces 32, like displays, storage media, input/output devices, etc.

The various components of the X-ray imaging system 10 are conventional and well known components. However, the computer system 30 is adapted to permit the X-ray imaging system 10 to operate and to implement methods in accordance with the present invention.

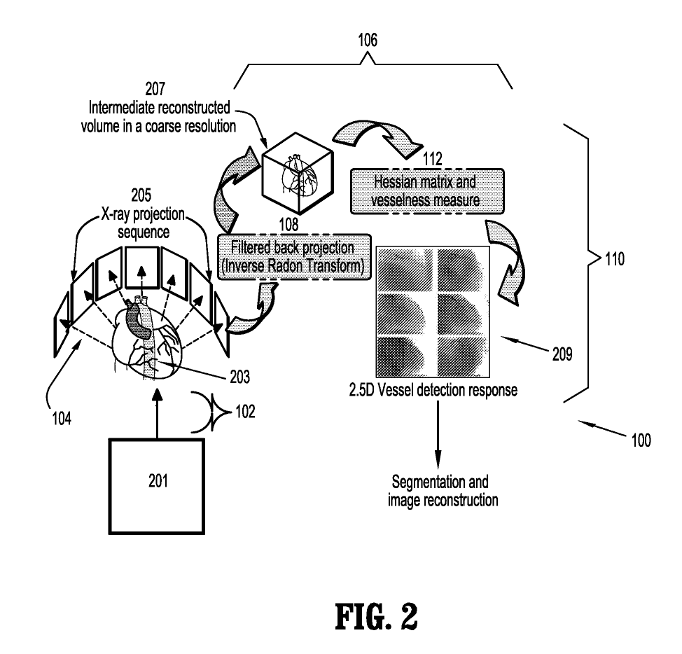

FIG. 2 is a schematic representation of a blood vessel detection (also known as extraction) method 100 implemented in accordance with the present invention. Initially, an X-ray imaging system 201 is used to acquire raw X-ray images of a patient and, more specifically, a coronary region of interest 203, such as the patient's heart and surrounding blood vessels. Diagnostic X-ray imaging is taken of the coronary area of interest 203 (Step 102) to ultimately visualize, for example, the coronary arteries, for the examining clinician. The method 100 may use various X-ray imaging systems 201 or techniques to perform the X-ray imaging, for example, a rotational X-ray imaging technique. The X-ray imaging is directed at the area of interest 203 from different origination points about the area of interest 203 to provide different angled views (Step 104). This produces a series of two-dimensional X-ray images 205 that is referred to as a 2D X-ray projection sequence. As noted above, the imaging is typically assisted by radio-opaque contrast agents delivered to a patient, usually during imaging (not shown). The blood vessels fill with contrast agent and therefore appear darker in the X-ray images 205 than the neighboring regions of the area of interest 203.

The contrast-enhanced images 205 (i.e., the representative image data signals) are processed by the associated computer systems, including image processors, of the X-ray imaging system 201 (Step 106). However, unlike prior methods, the method 100 provides a manner to exploit all available information to extract image features from the raw X-ray images 205 prior to all segmentation and reconstruction processes. In particular, the method 100 filters the back projection (i.e., the series of two-dimensional X-ray images 205) by applying an Inverse Radon Transform (IRT) on the 2D X-ray projection sequence (Step 108), which serves as the input signal. The IRT is a well-known mathematical expression and, like other transforms, provides an alternative mathematical representation of the images to the usual spatial domain representation. The frequency domain multiplication and addition processes of the IRT algorithm operate on the input signal to produce an intermediate image, specifically, an intermediate reconstructed volume 207 of the coronary area of interest 203 in a course resolution. The application of the IRT is equivalent to accumulating all back projected signals (images) and thus it recovers the original 3D image volume of the area of interest 203 from the angularly projected 2D images 205. In the Fourier domain, it is the same as summing up each individual volume which is merely reconstructed by one projected image.

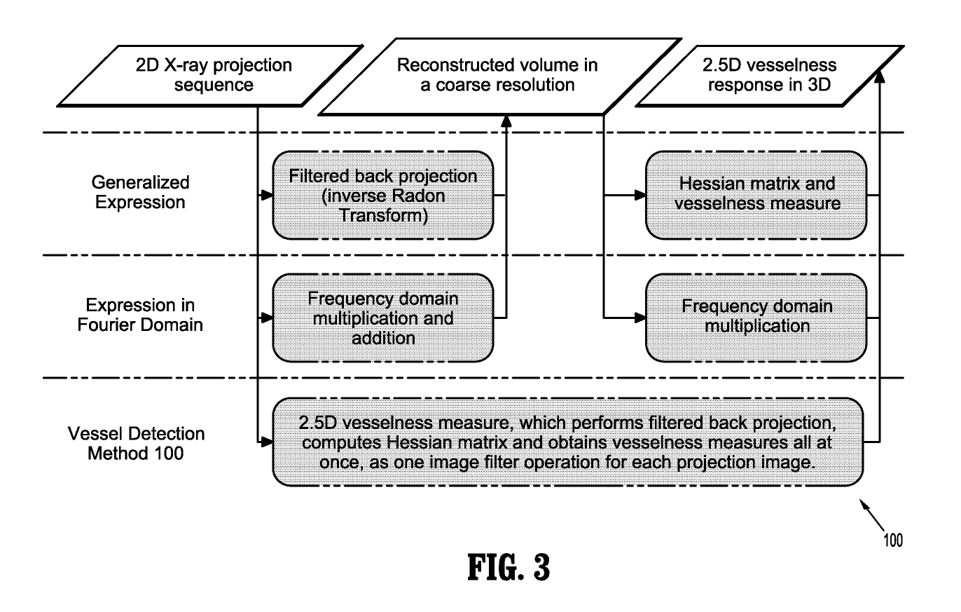

The method 100 then performs vesselness detection on the intermediate reconstructed 3D image volume 207 (Step 110) to acquire vesselness (or vessel detection) responses. To do so, the method 100 computes the well-known Hessian matrix, which describes local curvature and is based on the filtering responses of applying the second derivatives of Gaussian filters, and obtains vesselness measures (VM) (Step 112). However, since the analytical form of these filters is in the Fourier domain, the Fourier domain relationship between the IRT and Hessian matrix can be exploited and the IRT can be merged with the filters' Fourier expressions. The merged Fourier expression is thus considered as a set of Fourier domain-operated image filters and the IRT and the subsequent filtering process can be regarded as one filtering operation (if the input image signal is omitted). These image filters are particularly formed for the input back projected images 205, with their respective projection angles. Since they are formulated in between the 2D image inputs and 3D outputs, these filters are referred as a 2.5D vesselness measure and the method 100 thus obtains 2.5D vessel detection responses 209 in 3D. The method 100 employs, in effect, one image filter operation (Steps 108, 110, 112) for each projection image. FIG. 3 is a block diagram of the different representations of the described blood vessel detection method 100.

The method 100 replenishes information of correspondence between different image frames through the use of the 2.5D vesselness measure. Specifically, the 2.5D vesselness measure utilizes the projection matrices to realize the correspondence among different image frames to extract low level image features for segmentation and image reconstruction. Thus, the 2.5D vesselness measure can convey the image correspondence information to the subsequent processing steps.

Although the IRT is a well known technique that can capture correspondence between different image frames, it is not straightforward to perform IRT and subsequently vesselness detection in a conventional approach. The above-described method 100 of the present invention provides a novel way to utilize the IRT. Further, in performing the detection steps all at once as a merged operation, the method 100 provides several vital advantages to a conventional blood vessel detection/extraction approach.

First, the detection method 100 eliminates two Fourier transforms operations that would be required, and thus increases the efficiency and speed of the vessel detection process, by merging the two operations IRT and VM. This is possible in large part by the analytical form of the second derivatives of Gaussian functions and the filter used by the filtered-back projection. By merging their analytical forms, the method 100 completes the multiplication, the addition of frequency coefficients, and sampling all at once. In particular, a 2D Fast Fourier Transform (2D-FFT) is performed in preparing the data of the X-ray projection sequence 205 for the filtering operation. Without the method 100 of the present invention, a 2D Inverse Fast Fourier Transform (2D-IFFT) must be performed to reconstruct the intermediate volume 207 and a 3D-Fast Fourier Transform (3D-FFT) is required to compute the Hessian matrix and vesselness measure from the volume data. A 3D-Inverse Fast Fourier Transform (3D-IFFT) is performed to obtain the vessel detection response 209. In contrast, the method 100 of the present invention simply requires and performs the 2D-FFT and the 3D-IFFT operations (a single stage computation) and eliminates the intermediate 2D-IFFT and 3D-FFT operations (representing a two-stage computation). Consequently, the method 100 significantly reduces the computational cost (in terms of efficiency and speed) of the X-ray imaging system 201 to extract 3D vesselness features from 2D image frames.

Second, the detection method 100 reduces the numerical errors that can be incurred in the sampling processes. An X-ray imaging system 201 will normally require hundreds of image frames to effectively reconstruct a 3D volume of an imaged target. However, there are typically only a small number of image frames, for example, 4 to 10, available for coronary artery reconstruction. Since there is a severe lack of image frames to perform image reconstruction as well as vessel detection, avoiding or reducing numerical errors is a necessity. In the IRT operation, the usual rectangular grid coordinate system cannot match with the 2D rectangular image frames obtained in different projection angles. In such a case, interpolation of the back projection signals is widely applied to perform reconstruction of the image volume. However, obtaining the vesselness measure on interpolated signals is not preferable as the associated high pass filters (i.e., the second derivatives of the Gaussian functions) amplify noise and interpolation artifacts, as well as the numerical errors incurred in the intermediate 2D-IFFT and 3D-FFT operations. Further, factoring in the adverse effect of the limited number of image frames available for image reconstruction, it is impractical for the X-ray imaging system 201 to perform IRT and subsequently vesselness detection. In contrast, the detection method 100 performs the sampling process after all high-pass filtering operations. Although interpolation artifacts still exist, they are not amplified by high-pass filters operated in an earlier stage of the process. Consequently, the method 100 improves accuracy of the X-ray imaging system 201 by eliminating the intermediate 2D-IFFT and 3D-FFT operations and also makes practical performing IRT operations and subsequent vesselness detection.

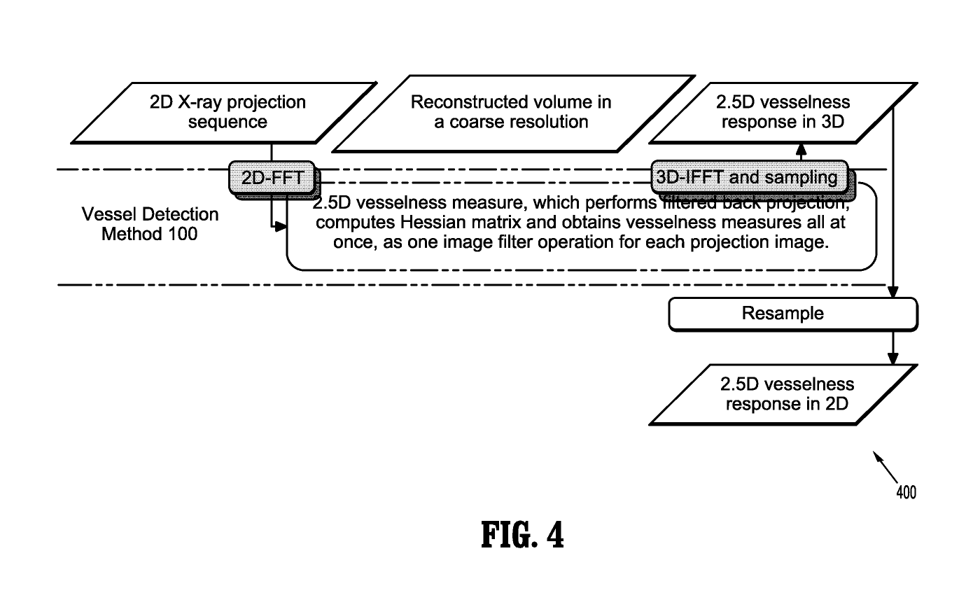

FIG. 4 is a block diagram of an alternative method 400 of blood vessel detection in accordance with the present invention. In addition to the detection steps of the previously described method 100, the alternative detection method 400 resamples the 2.5D vesselness detection response in 3D to acquire a 2.5D vesselness detection response in 2D for each reference frame. The resampling is done so that the responses match the 2D image resolution. The X-ray imaging system 201 uses the 2.5D vesselness detection response in 2D for subsequent 2D blood vessel segmentation.

In performing blood vessel segmentation on the resampled 2.5D vesselness detection responses in 2D, the alternative detection method 400 provides several advantages over blood vessel segmentation on 2.5D vesselness detection responses in 3D. First, the X-ray imaging system 201 in reconstructing the 3D vessels based on the 2D segmentation can use a coordinate system corresponding to the reference frame (i.e., the three axes of the reconstructed 3D volume correspond to the on-the-plane and the in-plane directions of the reference frame). In the sampling process involved in the earlier stage of the alternative detection method 400, the intermediate volume reconstruction 207 is also based on the coordinate system of the reference frame. Thus, the alternative detection method 400 avoids interpolation on the reference frame which, in turn, further refines the accuracy of the vesselness detection responses by avoiding interpolation on at least one image frame. Second, the method 400 permits the X-ray imaging system 201 to follow the original vessel detection/extraction routine to segment the vessels based on the 2.5D vesselness responses in 2D. In the original vessel detection/extraction routine, the correspondence among different image frames and the smoothness (such as, the vessel curvature and connectivity) of the detection results are simultaneously considered. This is not available to the X-ray imaging system 201 in performing segmentation in the 2.5 vesselness measure in 3D.

Note that the methods provided by the present invention are not bound to any particular interpolation technique and can work well with all standard interpolation techniques such as bilinear/bicubic interpolation, spline interpolation, nearest neighbor and Gaussian interpolation.

Other modifications are possible within the scope of the invention. For example, the subject to be scanned may be an animal subject or any other suitable object rather than a human patient. Also, the X-ray imaging system 10 has been described in a simplified fashion and may be constructed in various well-known manners and using various well-known components. For example, the computer system 30 may incorporate the control portions of the various imaging system 10 components or may be modularly constructed with separate but coordinated units, such as an image processing unit, user interfaces, workstations, etc. Also, although the steps of each method have been described in a specific sequence, the order of the steps may be re-ordered in part or in whole and the steps may be modified, supplemented, or omitted as appropriate.

Also, the imaging system 10 and the computer system 30 may use various well known algorithms and software applications to implement the processing steps and substeps, such as segmentation, image reconstruction, etc. Further, the 2.5D vesselness measure may be implemented in a variety of algorithms and software applications, for example, VC++6 incorporated in a proprietary prototyping framework based on OpenInventor. Further, the 2.5D vesselness detection responses may be obtained based on either filtered-back-projected IRT or plain IRT operations. Further, the methods 100, 400 of the present invention may be supplemented by additional processing steps or techniques to remove resulting image artifacts, provide a sufficient number of image frames, or, otherwise, insure reliable blood vessel image reconstruction.

What is claimed is:

1. A method of reconstructing 3D images of vascular structures, comprising:
a. obtaining 2D X-ray projection images of the vascular structures to be imaged;
b. extracting image features from the X-ray images via the use of a 2.5D vesselness measure;
c. segmenting the vascular structures from the X-ray images using the extraction results; and
d. reconstructing 3D images of the vascular structures from the segmentation results.

2. The method of claim 1, wherein the vascular structures comprise coronary arteries.

3. The method of claim 1, wherein the extracting step is performed before the segmenting and the reconstructing steps.

4. The method of claim 3, wherein the extracting step comprises applying an inverse radon transform on the X-ray images and performing vesselness detection to acquire vesselness responses.

5. The method of claim 4, wherein the applying and performing steps are performed as one merged operation.

6. The method of claim 5, wherein the performing step comprises performing vesselness detection to acquire vesselness detection responses in 3D and the method further comprises resampling the vesseleness detection responses in 3D to acquire a vesselness detection response in 2D for each reference image frame, said segmenting step segmenting the vascular structures from the X-ray images using the resampling results.

7. The method of claim 4, wherein the performing step comprises computing a Hessian matrix and obtaining vesselness measures using the inverse radon transform results.

8. The method of claim 7, wherein the applying and performing steps are performed as one merged operation.

9. The method of claim 8, wherein the performing step comprises performing vesselness detection to acquire vesselness detection responses in 3D and the method further comprises resampling the vesseleness detection responses in 3D to acquire a vesselness detection response in 2D for each reference image frame, said segmenting step segmenting the vascular structures from the X-ray images using the resampling results.

10. The method of claim 3, wherein the extracting step comprises accumulating all the 2D X-ray projection images and performing vesselness detection on the accumulation results to acquire vesselness detection responses.

11. The method of claim 10, wherein the applying and performing steps are performed as one merged operation.

12. The method of claim 11, wherein the performing step comprises performing vesselness detection to acquire vesselness detection responses in 3D and the method further comprises resampling the vesseleness detection responses in 3D to acquire a vesselness detection response in 2D for each reference image frame, said segmenting step segmenting the vascular structures from the X-ray images using the resampling results.

13. A method of coronary artery 3D reconstruction, comprising:
a. obtaining a 2D X-ray projection sequence of a coronary artery to be imaged; and
b. filtering each projection image of the back projection for the 2D X-ray projection sequence using a vesselness measure that realizes the correspondence among different image frames to extract low level image features for subsequent segmentation and image reconstruction of the coronary artery.

14. The method of claim 13, wherein the filtering step comprises performing a merged operation of an inverse radon transform and a vesselness detection.

15. The method of claim 13, wherein the filtering step comprises performing a merged operation of a filtered back-projected inverse radon transform and a vesselness detection.

16. The method of claim 13, wherein the filtering step comprises performing a merged operation of an inverse radon transform, a Hessian matrix computation, and a vesselness measure.

17. The method of claim 13, further comprising resampling the filtering results to acquire a vesselness detection response in 2D for each reference image frame for subsequent 2D segmentation.

18. A method of blood vessel extraction for rotational angiographic X-ray sequences, comprising obtaining a 2.5D vesselness detection response in 3D.

19. The method of claim 18, wherein the obtaining step comprises utilizing the projection matrices to realize the correspondence among different image frames to extract low level image features for subsequent segmentation and 3D image reconstruction.

20. A 3D X-ray imaging system, comprising an X-ray source that generates X-ray beams; an X-ray detector that is adapted to receive the X-ray beams; a support table positioned between the X-ray source and the X-ray detector such that the X-ray beams pass through a portion of the vasculature structure of a subject lying thereon and project onto the X-ray detector, said detector converting the raw X-ray projections into image data signals for subsequent processing; and a computer system which controls the operation of the system and its components and processes the image data obtained from the X-ray detector to transform them into a reconstructed volumetric image of the imaged portion of the vasculature structure for display, storage, and/or other usage, said computer system filtering each projection image of the back projection for the X-ray images using a vesselness measure that realizes the correspondence among different image frames to extract low level image features for subsequent segmentation and 3D image reconstruction of the imaged portion of the vasculature structure.

21. The system of claim 19, wherein the system comprises a rotational X-ray apparatus whereby the X-ray source and the X-ray detector are mounted on opposite ends of, and coupled to one another via, a rotatable C-arm gantry arrangement that moves the X-ray source and the X-ray detector about the person and the table in a coordinated manner so that the X-ray projections of the imaged portion of the vasculature structure can be generated from different angular directions and a series of 2D X-ray projections are acquired along an arced path.
